(19) United States
(12) Patent Application Publication
(10) Pub. No.: US 2019/0059749 A1
(43) Pub. Date: Feb. 28, 2019

(54) SMARTGRIP BIOMETRIC ELECTRONIC TABLET STAND

(71) Applicant: HOMEWARD HEALTH, LLC, Toledo, OH (US)

(72) Inventor: Joseph W Gough, Whitmore Lack, MI (US)

(73) Assignee: CareVoice Inc, Toledo, OH (US)

(21) Appl. No.: 15/765,259

(22) PCT Filed: Jul. 18, 2016
(86) PCT No.: PCT/US16/42713

§ 371 Date: Mar. 31, 2018

Related U.S. Application Data
(60) Provisional application No. 62/194,668, filed on Jul. 20, 2015.

(51) Int. Cl. A61B 5/0205; G06F 1/16; G16H 50/20; A61B 5/22; A61B 10/00; A61B 5/16; A61F 13/38; G06F 3/01; G01N 33/50

(52) U.S. Cl. CPC A61B 5/02055; G06F 1/1626; G16H 50/20; A61B 5/225; A61B 5/0816; A61B 5/165; A61F 13/38; G06F 3/011; G01N 33/50; A61B 10/0051

(57) ABSTRACT

A biometric electronic tablet device for a user includes a base platform adapted to enclose an electronic tablet for the user and a plurality of legs extending from the base platform and downwardly and adapted to contact a support surface to elevate the base platform and the electronic tablet over the user. The biometric electronic tablet stand includes biometric sensors for acquiring a grip strength and biometric data of a user.

### TECHNICAL FIELD

The present invention relates generally to patient healthcare, in the form of an adjustable biometric measuring device that encloses, holds, and interacts with an existing electronic device such as a tablet to allow the patient and others to obtain a wide variety of medical and healthcare information in real time.

### BACKGROUND

The global stress on qualified healthcare staffing has reached a tipping point of dangerous consequences. In order to meet the rising demand of an aging population with complex chronic illnesses, agile patient-directed technology must be deployed to alleviate increasing pressure on taxed human resources. Thus, there is a need in the art to provide a device with an integrated software system that helps transmit information about patients in the simplest and most effective possible way.

### SUMMARY OF THE INVENTION

Accordingly, the present invention is an adjustable biometric measuring device (“SmartGrip”) that integrates with an existing tablet and includes a base platform, adapted to enclose the tablet, and which has a plurality of legs extending from the base platform downwardly and adapted to contact a support surface to elevate the base platform and the electronic tablet over the patient.

The adjustable biometric device further includes sensors for measuring biometric data of a patient including grip strength, vital signs and stress levels (i.e., diastolic and systolic blood pressure, blood oxygen levels, respiration rate, pulse rate and/or temperature), blood glucose levels, blood chemistry (hematocrit, complete blood count, hemoglobin), blood hormones, user's frailty, and other biometric measurements (view the user or user identification). Once the data is acquired, the-battery powered device transmits it into a risk stratifying algorithm which integrates with the tablet software to graphically display data and risk factors in real time. If the data meets pre-defined markers, alert messages are automatically generated and distributed to the user or user's caregiver.

One advantage of the present invention is that a new adjustable biometric device (that uses and works with almost any electronic tablet) is provided for a user such as a medical patient. Another advantage of the present invention is that it is a flexible multi-point elevated device that encloses and protects an electronic tablet while having the capacity to acquire the grip strength and a great deal of other biometric data of a user. An additional advantage of the present invention is that the biometric device encloses, protects, and powers an electronic tablet while acquiring and integrating sensor-accessible biometric data with psychosocial survey data, producing real-time physical and psychosocial analytics and risk stratification.

Other features and advantages of the present invention will be readily appreciated, as the same becomes better understood, after reading the subsequent description taken in conjunction with the accompanying drawings.

### BRIEF DESCRIPTION OF THE DRAWINGS

FIG. 1 is a perspective view of one embodiment of a biometric electronic tablet device.

### DETAILED DESCRIPTION OF THE INVENTION

### Definitions

The term “electronic tablet” or “tablet computer,” commonly shortened to tablet, is a handheld mobile computer with a touchscreen display, circuitry, software, and battery in a single device. Electronic tablets have built-in sensors, including cameras, a microphone, and speakers. The term “tablet” can also be used to describe a smartphone or any other handheld mobile computer that has the same or similar capabilities as an electronic tablet.

The term “biometric sensors” means sensors capable of detecting patient images, vital signs as well as stress level indications.

The term “glucometer” is a medical device for determining the approximate concentration of glucose in the blood.

The term “capacimeter” or “frailty instrument” refers to a medical device for measuring clinimetric properties indicating frailty of the user.

The term “photonic technology” involves integrations for biometric measurements such as a camera-enabled biometric reader.

The term “cytometer” refers to a medical device used to analyze the physical and chemical characteristics of a fluid, such as a sample from a mouth swab of the user.

### Adjustable Biometric Measuring Device

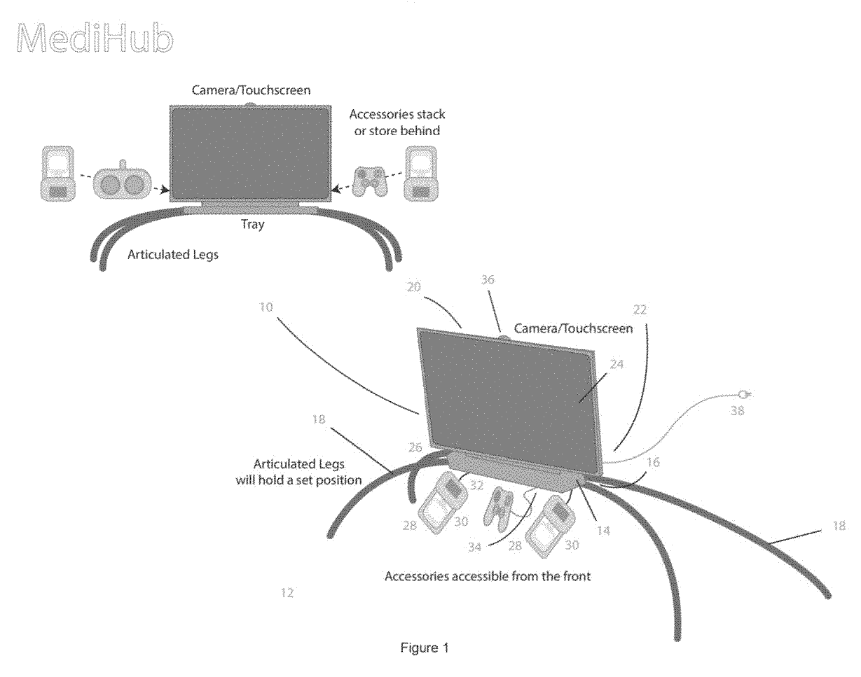

Referring to FIG. 1, one embodiment of an adjustable biometric measuring device that integrates with an electronic tablet, according to the present invention, is shown at 10 for a user such as a medical patient, generally indicated at 12. The device 10 includes a base platform 14 having a slot or groove 16 therein. The device 10 also includes one or more device legs 18 extending from the base platform 14 and downwardly to elevate the base platform 14 over the user 12, illustrated in a lying down position below the stand 10. In the embodiment illustrated, two legs of the device 18 are disposed in the groove 16 and extend from each lateral end of the base platform 14 in an articulate manner to engage a support surface to elevate the base platform 14 above the support surface. In one embodiment, the base platform 14 is connected to the device legs 18 and these device legs 18 extend from the base platform 14, wrapping around the waist, upper legs, or midsection of the user 12. The device legs 18 are made of flexible memory material or composites shaped like extendable legs with metallic or plastic cylinder coverings to provide stability and integrity. The device legs 18 have a flexible extension to create a table effect using their multi-point configuration to wrap around the user 12, or adjusting to stand up on any solid surface. It should be appreciated that the device legs 18 may be used for standing platform use and folding storage applications. It should be appreciated that the device legs 18 are adjustable.

The device 10 includes a tablet enclosure 20 for enclosing an electronic tablet 22 for tablet protection. The tablet enclosure 20 is generally rectangular in shape. The tablet enclosure 20 includes a screen shield or protective covering 24 for the electronic tablet 22. The tablet enclosure 20 expands and retracts to allow for multiple size electronic tablets 22 to be locked into a protective covering and affixes to the adjustable base platform 14. The tablet enclosure 20 also includes a locking mechanism 26 to keep the tablet enclosure 20 from being opened by public users. It should be appreciated that the legs 18 allow for the tilting and rotating of the tablet enclosure 20. It should also be appreciated that the screen shield 24 is for biohazard protection. It should further be appreciated that the electronic tablet 22 is conventional.

The device 10 may include one or more dynamometer or grip strength sensors 28 that are adapted to be gripped by the user 12 to sense the grip strength of the user 12. The sensors 28 may be integrated around one or more handles 30 to allow grip test measurements to be taken and integrated into the software in real time. It should be appreciated that the dynamometer is for grip strength measurement. It should also be appreciated that the sensor handles 30 are for measurements and ease of use of the device 10.

The device 10 may also include one or more biometric sensors 32 for vital signs of the user 12. In one embodiment, the biometric sensors 32 are affixed to the handles 30 placed on the upper half of the device legs 18 closest to the base platform 14. These handle sensors 32 can detect a variety of biometric vital signs, as well as stress level indications. The device 10 may include one or more glucometer and photonic technology integrations such as connections or ports 34. The device 10 may also include a camera-enabled biometric reader 36 to view the user 12. The biometric sensors 32 are measurement tools such as glucometers, capacimeters, cytometers, chemical and genomic testing applications that can be integrated into the base platform 14 and fed into software of the electronic tablet 22 for evaluation on a dashboard of the electronic tablet 22. It should be appreciated that the biometric sensors 32 are for vital sign and stress level measurement, the glucometer is for blood glucose measurement, the capacimeter is for frailty indication measurement, the photonic technology integrations are for biometric measurements, and the cytometer and chemical testing applications are for mouth swab or fluid testing. It should also be appreciated that full tablet integration with data transfer from the sensors and digital testing applications into software platform and machine learning algorithms produce real time assessments and evaluations blended with interactive cognitive data acquisition.

The device 10 may include a charging cord 38 for charging the electronic tablet 22. The device 10 may also include battery life storage 40 for batteries (not shown). In one embodiment, the battery life storage 40 and charging components are built into the base platform 14 and the legs 18 for easy plugging into outlets and long term use without charging. It should be appreciated that the device 10 provides long battery life and easy charging.

In operation, the device 10 is a flexible multi-point device to enclose and protect the electronic tablet 22 while acquiring the grip strength and biometric data of the user 12. Once the data is acquired, the battery-powered device 10 transmits it into a risk stratifying algorithm which graphically displays the data and risk factors in real time. If the data meets pre-defined markers, alert messages are automatically generated and transmitted to a caregiver (not shown) of the user 12. Likewise, the users 12 themselves can access the same analytic profile to self-assess their needs. The device 10 can also detect stress levels on an individualized question basis indicating the probability that the user 12 may not be giving honest answers to the questions.

Accordingly, the present invention provides a device 10 that delivers digital multimedia content and software programs to the bedside or waiting room environments, while simultaneously using sensors, dynamometers, and other measurement tools to feed real-time biometric data from the user 12 into the software and machine learning algorithms. Once the data is acquired, the battery-powered device 10 transmits the data into risk stratifying assessment programs, which graphically display the data in real time.

The present invention has been described in an illustrative manner. It is to be understood that the terminology which has been used is intended to be in the nature of words of description rather than of limitation.

Many modifications and variations of the present invention are possible in light of the above description. Therefore, the present invention may be practiced other than as specifically described.

1. An adjustable biometric measuring device for a user, comprising: a base platform adapted to enclose and interact with an electronic tablet; and a plurality of legs extending from said base platform and downwardly, adapted to contact a support surface to elevate said base platform and the electronic tablet over the user.

2. The biometric measuring device as set forth in claim 1 including a tablet enclosure adapted for enclosing the electronic tablet.

3. The biometric measuring device as set forth in claim 2 wherein said tablet enclosure expands and retracts to allow for multiple size electronic tablets.

4. The biometric measuring device as set forth in claim 2 including a locking mechanism to keep said tablet enclosure from being opened by public users.

5. The biometric measuring device as set forth in claim 2 wherein said tablet enclosure includes a screen shield for biohazard protection of the electronic tablet.

6. The biometric measuring device as set forth in claim 1 including one or more biometric sensor(s) that are adapted to be gripped by the user to sense the grip strength of the user.

7. The biometric measuring device as set forth in claim 1 including one or more biometric sensor(s) adapted for sensing vital signs including diastolic and systolic blood pressure, blood oxygen levels, respiration rate, pulse rate, blood chemistry, blood glucose, temperature, and/or frailty of the user.

8. The biometric measuring device as set forth in claim 7 wherein said one or more biometric sensor(s) comprise a glucometer for blood glucose measurement of the user.

9. The biometric measuring device as set forth in claim 7 wherein said one or more biometric sensor(s) comprise a capacimeter for frailty indication measurement of the user.

10. The biometric measuring device as set forth in claim 7 wherein said one or more biometric sensors comprises photonic technology integrations for biometric measurements of the user.

11. The biometric measuring device as set forth in claim 7 wherein said one or more biometric sensors comprise cytometer and chemical testing devices for sample of mouth swab or fluid testing of the user.

12. The biometric measuring device as set forth in claim 1 wherein said legs are adjustable.

13. The biometric measuring device as set forth in claim 1 wherein said legs are made of a flexible material.

14. The biometric measuring device as set forth in claim 1 further comprising charging components and/or battery storage components built into the base platform and/or legs for plugging into electrical outlets and/or long-term use without charging.

15. The biometric measuring device as set forth in claim 1 further comprising electronic tablet integration with data transfer from biometric sensors and digital testing applications into a software platform and machine-teaming algorithms to produce real-time user assessment.

16. A method of determining one or more biometric measure(s) in a user comprising a biometric measuring device comprising a base platform adapted to enclose an electronic tablet for the user, one or more biometric sensors attached to base platform and/or legs, and a plurality of legs extending from said base platform downwardly adapted to contact a support surface to elevate said base platform and the electronic tablet over the user.

17. The method of claim 16 wherein the one or more biometric sensors is used to obtain in real time one or more biometric measure(s).

18. The method of claim 17 wherein the one or more biometric measures are selected from hand grip strength, vital signs and stress levels (i.e., diastolic and systolic blood pressure, blood oxygen levels, respiration rate, pulse rate and/or temperature), blood glucose levels, blood chemistry (hematocrit, complete blood count, hemoglobin), blood hormones, user's frailty, and biometric measurements (view the user or user identification).

25. The method of claim 16 wherein the biometric sensor is selected from a dynamometer, a glucometer, a photonic technology integrations, camera-enabled biometric reader, capacimeter, cytometer, and chemical testing applications.

26. The method of claim 25 wherein the biometric sensor is a dynamometer used to measure hand grip strength.

27. The method of claim 25 wherein the biometric sensor is a glucometer used to measure blood glucose levels.

28. The method of claim 25 wherein the photonic technology integrations (camera-enabled biometric reader) is used to view the user or user identification.

29. The method of claim 25 wherein the capacimeter is used to measure user frailty.

30. The method of claim 25 wherein the cytometer is used for fluid testing.

31. The method of claim 25 wherein the chemical testing
